(19) United States
(12) Patent Application Publication
(10) Pub. No.: US 2006/0167280 A1
(43) Pub. Date: Jul. 27, 2006

(54) Method for purifying simvastatin

(76) Inventors: Jung Woo Kim, Kangseo-gu (RU); Tae Won Kang, Seoul (RU); Eui Seok Park, Yonghwajugong (RU); Dong Ock Cho, Su-won (RU); Hyeung Geun Baek, Doojeong-dong (RU)

Correspondence Address: Peter F. Corless;EDWARDS & ANGELL, LLP, P.O. Box 55874, Boston, MA, 02205, US

(21) Appl. No.: 10/503,769

(22) PCT Filed: Feb. 6, 2003
(86) PCT No.: PCT/KR03/00257

§ 371 Date: Apr. 20, 2005

(30) Foreign Application Priority Data
Feb. 8, 2002 (KR) 10-2002-0007235

(51) Int. Cl. C07D 309/30

(52) U.S. Cl. 549/292

(57) ABSTRACT

The present invention relates to a purification method of simvastatin crystal, more particularly to a method of effectively removing impurities contained in crude simvastatin obtained by a conventional synthetic method of simvastatin while minimizing loss of effective components to obtain high-quality simvastatin with good yield.

### TECHNICAL FIELD

The present invention relates to a purification method of simvastatin crystal, more particularly to a method of effectively removing impurities contained in crude simvastatin obtained by using a conventional synthetic method of simvastatin while minimizing loss of effective components to obtain high-quality simvastatin with good yield.

### BACKGROUND ART

Simvastatin, a substance represented by the following Chemical Formula 1, is known to cure hyperlipidemia due to its superior inhibitory activity against HMG Co-A reductase.

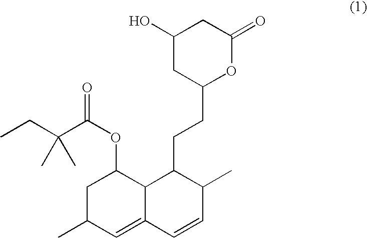

Generally, simvastatin is synthesized by a series of reactions comprising hydrolysis, lactonization, alcohol protection of the lactone ring, acylation and deprotection, by using lovastatin as a starting material. A variety of preparation methods with improvements in each step of the synthesis have been disclosed [U.S. Pat. No. 4,444,784; Korean Patent Publication No. 2000-15179].

As mentioned above, there have been known many methods for preparing simvastatin, and the kinds and contents of impurities contained in crude simvastatin are known to vary depending upon the preparation method used. However, typical impurities contained in crude simvastatin are substances represented by the following Chemical Formulas 2a, 2b and 2c.

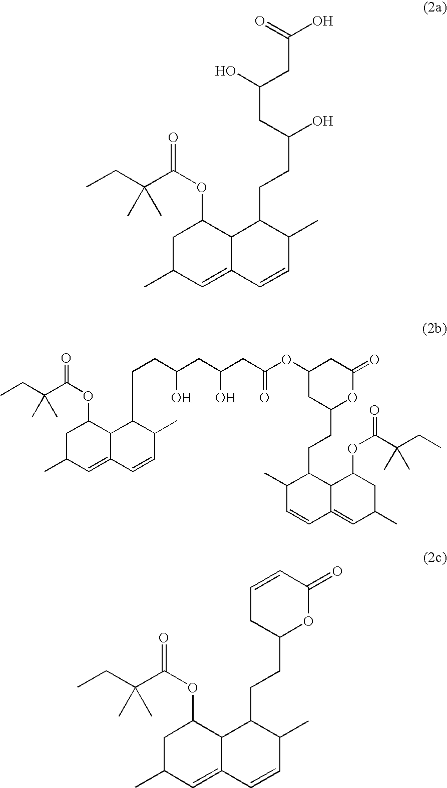

The impurity represented by Chemical Formula 2a is generated by opening of the lactone ring; the impurity represented by Chemical Formula 2b is a dimer generated by esterification of 3-hydroxylactone and free acid; and the impurity represented by Chemical Formula 2c is anhydrosimvastatin with the 3-hydroxy of 3-hydroxylactone filtered. In particular, the dimer represented by Chemical Formula 2b is known very difficult to remove even by the most precise and sophisticated recrystallization.

Korean Patent No. 133,599 discloses a method of reducing content of the dimer represented by Chemical Formula 2b to a level below 0.2%. However, the above patent controls generation of the dimer during the process of simvastatin preparation, and it is totally different from the present invention, which separates and removes the dimer from crude simvastatin. Moreover, the above patent uses 1.2 to 1.5 equivalents of strong acid during lactonization, and requires an additional step to neutralize the strong acid after the reaction is completed. Considering the difficulty in handling of strong acid, environmental effect and complicatedness of preparation steps, the above patent is limited in applying it to mass production of an industrial scale.

It may be effective to obtain simvastatin with high-purity by minimizing impurities by controlling the method of simvastatin synthesis. However, in view of mass production of an industrial scale, it will be much more effective to obtain simvastatin with high-purity by separating and removing impurities contained in crude simvastatin, irrespective of the synthesis method being used.

Accordingly, an object of the present invention is to provide a method of purifying simvastatin by effectively separating and removing impurities contained in crude simvastatin.

### DISCLOSURE OF INVENTION

The present invention relates to a method of purifying crude simvastatin, which comprises:

(a) a step of concentrating crude simvastatin by dissolving in a solvent, adding simvastatin seed in said concentrate for partial crystallization and adding hexane or cyclohexane to obtain Crystal #1;

(b) a step of suspending said Crystal #1 in alkyl acetate and adding hexane or cyclohexane to obtain Crystal #2; and

(c) a step of dissolving said Crystal #2 in a solvent selected from a group consisting of dimethoxyethane, tetrahydrofuran, 1,4-dioxane, dimethylether and dimethoxypropane and adding purified water to obtain purified simvastatin crystal.

Hereinafter, the present invention is described in more detail.

The present invention relates to a method for obtaining high-purity simvastatin crystal by effectively removing impurities contained in crude simvastatin obtained by a conventional synthetic method.

Any crude simvastatin obtained by a conventional synthetic method can be used in the present invention. In particular, the present invention provides an excellent method for removal of such impurities as the one generated by opening of the lactone ring (represented by Chemical Formula 2a), a dimer represented by Chemical Formula 2b, anhydrosimvastatin represented by Chemical Formula 2c, and a mixture of these.

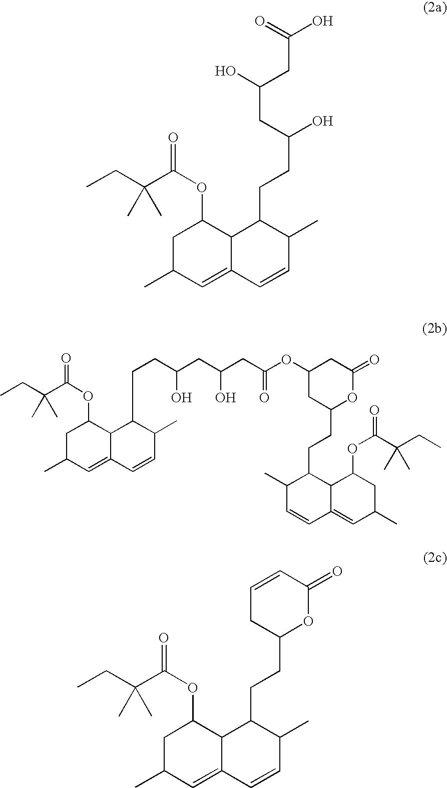

In a conventional preparation method of simvastatin, the impurity generated by opening of the lactone ring (represented by Chemical Formula 2a) may be contained over 3.0%, and more precisely in the amount of 3.0 to 5.0%; the dimer represented by Chemical Formula 2b may be contained over 0.6%, and more precisely in the amount of 0.6 to 1.0%; and anhydrosimvastatin represented by Chemical Formula 2c may be contained over 1.0%, and more precisely in the amount of 1.0 to 2.0%. These impurities may be contained alone or in combination of two or three components. Also, depending upon the preparation method, triphenylphosphine oxide may be contained as an impurity over 2%, and more precisely in the amount of 2.0 to 3.0%, which can be also effectively removed by the method of the present invention.

Each step of the present invention is described in greater detail hereunder.

The step of obtaining Crystal #1 is as follows. Crude simvastatin is dissolved in an organic solvent. This solution is concentrated at a temperature ranging from 0 to 40° C., more preferably at a temperature of from 0 to 15° C. For the organic solvent, methylene chloride, chloroform, tetrahydrofuran, ethyl acetate, etc. may be used. The concentrated solution is seeded with a trace amount of simvastatin for partial crystallization. Then, hexane or cyclohexane is added for complete crystallization. The crystallized solution is stirred at −10 to 30° C. for from 30 minutes to 3 hours and filtered to obtain Crystal #1.

The step of obtaining the Crystal #1 is characterized by the seeding process. If the solution is directly filtered without seeding, the solution should be heated to increase the filtering efficiency. However, the increase in temperature causes the increase in content of the dimer represented by Chemical Formula 2b. Also, with regard to addition of a nonpolar solvent such as hexane or cyclohexane, filtering after crystallization becomes very difficult unless seeding is carried out. The Crystal #1 obtained by carrying out the seeding process according to the present invention contains quite reduced amount of the impurity generated by opening of the lactone ring (represented by Chemical Formula 2a). Also, the dimer represented by Chemical Formula 2b and anhydrosimvastatin represented by Chemical Formula 2c are removed significantly. Moreover, industrialization is made possible by resolving the problems of crystallization and filtration.

The next step of obtaining Crystal #2 is as follows. The Crystal #1 is suspended in alkyl acetate such as ethyl acetate, isopropyl acetate, butyl acetate or isobutyl acetate. Then, hexane or cyclohexane is added and the temperature is maintained in the range of from 0 to 40° C. If the temperature of the solution is below 0° C., filtration becomes insufficient due to incomplete crystallization. In contrast, if it exceeds 40° C., the content of the dimer increases. Then, the solution is left for from 30 minutes to 2 hours at 0 to 5° C. The solution is stirred for from 30 minutes to 4 hours in the same temperature range, and filtered to obtain Crystal #2.

This step of obtaining Crystal #2 significantly removes triphenylphosphine oxide. Also, the dimer represented by Chemical Formula 2b and anhydrosimvastatin represented by Chemical Formula 2c are removed significantly.

The last step of obtaining the final simvastatin crystal is as follows. The Crystal #2 is completely dissolved in a solvent, such as dimethoxyethane, tetrahydrofuran, 1,4-dioxane, dimethylether or dimethoxypropane. Then, active carbon is added to the solution, and the solution is filtered at 15 to 30° C. for from 30 minutes to 2 hours, more preferably, at 20 to 25° C. for from 30 minutes to 1 hour while stirring. The filtration process using active carbon is an optional step. By this process, color of simvastatin is improved, and impurities other than those mentioned above, such as some heavy metals, are removed effectively. Purified water is slowly added to the filtrate at 10 to 30° C. for from 30 minutes to 3 hours. This solution is stirred at the same temperature for from 1 to 3 hours and filtered to obtain purified simvastatin.

The present invention is also characterized by selection of solvents that dissolve and grow the crystal. Particularly, the present invention doest not use any alcoholic solvent. If an alcoholic solvent is used, other impurities such as alkyl ester may be generated. The present invention uses hexane or cyclohexane as a crystallization solvent for obtaining Crystal #1 and Crystal #2, and purified water for obtaining the final crystal. These solvents prevent coagulation during the crystal growth and remove some water-soluble inorganic salts. Therefore, they increase efficiency and purity and readily industrialize the purification process of simvastatin.

The final simvastatin crystal obtained by the present invention contains less than 0.1% of the impurity generated by opening of the lactone ring (represented by Chemical Formula 2a), less than 0.4% of the dimer represented by Chemical Formula 2b, and less than 0.4% of anhydrosimvastatin represented by Chemical Formula 2c. More specifically, it contains from 0.05 to 0.1% of the impurity generated by opening of the lactone ring (represented by Chemical Formula 2a), from 0.2 to 0.4% of the dimer represented by Chemical Formula 2b, and from 0.1 to 0.4% of anhydrosimvastatin represented by Chemical Formula 2c.

Also, the present invention minimizes loss of simvastatin during the purification. The purity of simvastatin crystal obtained by the present invention was over 99% and was significantly higher than that of crude simvastatin.

Also, if the solution remaining after purification is collected and used in the step of obtaining Crystal #1, the total yield can be improved by from 5 to 10%.

The present invention is explained in more detail based on the following Examples but they should not be construed as limiting the scope of this invention.

### BRIEF DESCRIPTION OF DRAWINGS

FIG. 1 shows a result of HPLC analysis for crude simvastatin used in Example 1.

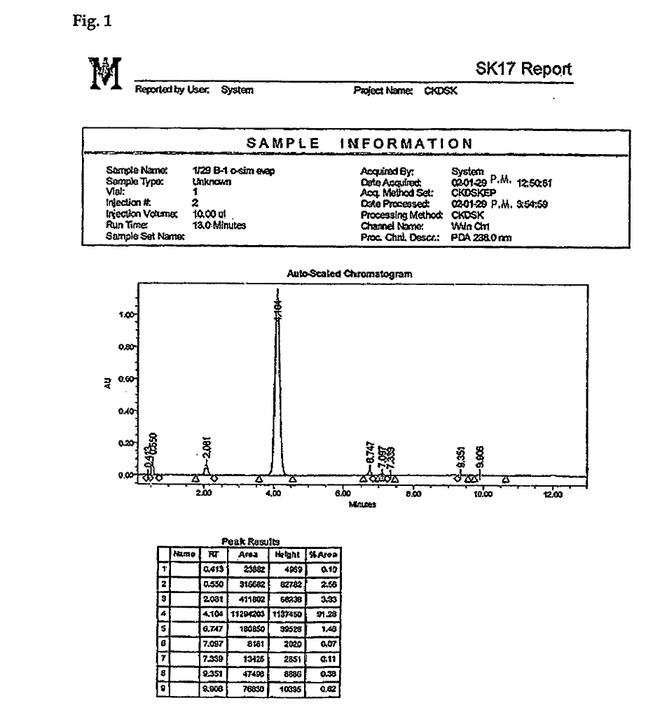

FIG. 2 shows a result of HPLC analysis for Crystal #1 obtained in Example 1.

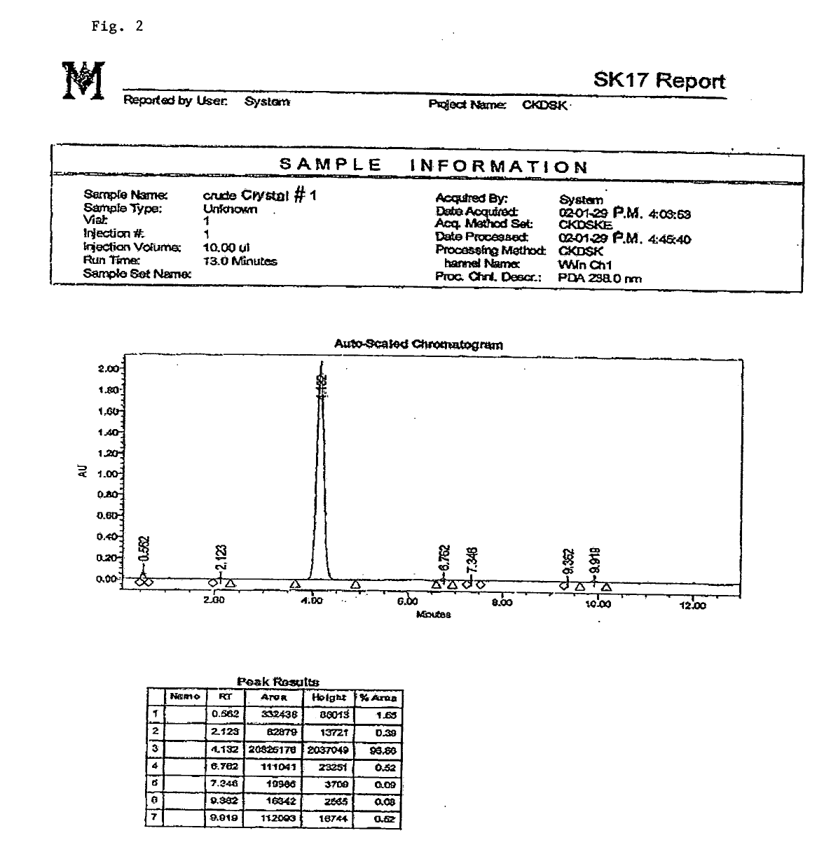

FIG. 3 shows a result of HPLC analysis for Crystal #2 obtained in Example 1.

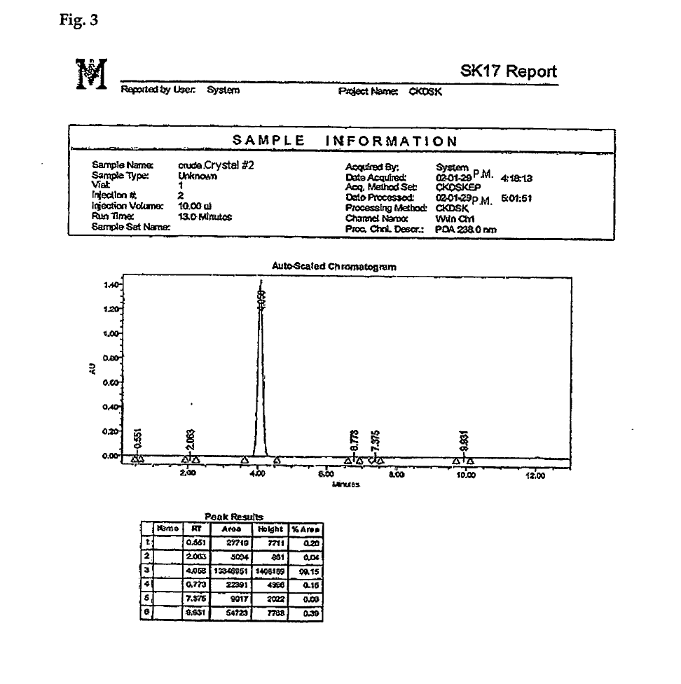

FIG. 4 shows a result of HPLC analysis for the final simvastatin crystal obtained in Example 1.

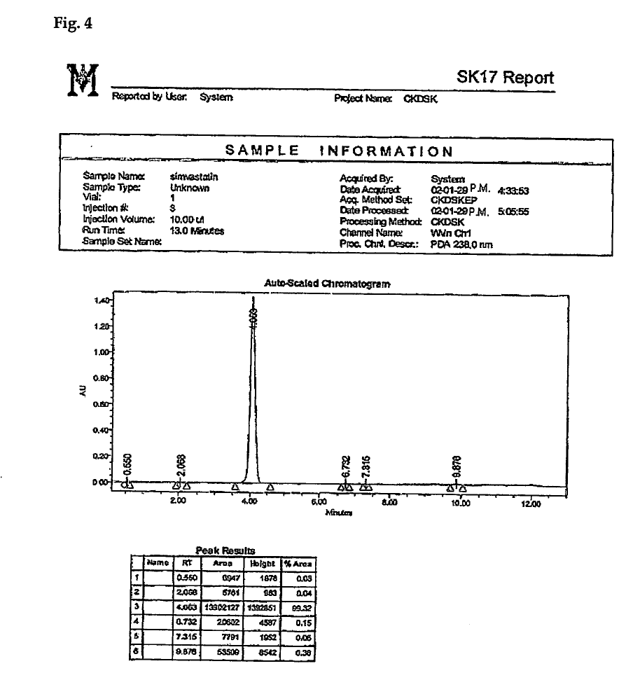

### EXAMPLE

### Example 1

10 g of crude simvastatin (purity: 91.28%) was dissolved in 650 ml of methylene chloride and completely concentrated at about 15° C. During the concentration, simvastatin was added in as a seed for crystallization. 3000 ml of hexane was added for 30 minutes while vigorously stirring the solution. Then, the solution was slowly cooled to about 5° C. for 1 hour. At the same temperature (about 5° C.), the solution was stirred for 1 hour, and then filtered. The filtrate was washed with hexane and dried to obtain 93.3 g of Crystal #1.

The obtained 93.3g of Crystal #1 was suspended in 280 ml of ethyl acetate. Then, 1400 ml of hexane was dropped for 30 minutes and the temperature was raised to 30° C. The solution was cooled to 5° C. for 1 hour, stirred at the same temperature for 1 hour, and then filtered. The filtration mother liquor was collected and dried. The crystal was washed with hexane and dried to obtain 83.7 g of Crystal #2.

The obtained 83.7 g of Crystal #2 was dissolved in 350 ml of dimethoxyethane. After adding 4.3 g of active carbon, the solution was stirred at 20° C. for 30 minutes and filtered. The filtrate was washed with 350 ml of dimethoxyethane and 1300 ml of water was dropped for 1 hour at 20° C. After stirring for 1 hour at the same temperature, it was washed with 1700 ml of water. Then, it was washed with 880 ml of hexane and dried to obtain 82.0 g of high-purity simvastatin crystal (purity: 99.32%).

Solutions remaining in the first, second and third purification steps were collected and concentrated. This solution was placed under the same purification process as shown above to further obtain 5 g of simvastatin crystal (purity: 99.13%).

Each crystal obtained in each crystallization step was analyzed with an HPLC (Waters; 2690). The content analysis result is shown in the following Table 1.

TABLE 1

| | | | Impurities | | | | Yield | |
|---|---|---|---|---|---|---|---|---|
| | | | | Chemical | Chemical | Chemical | By | |
| | Simvastatin | | Triphenylphosphine | Formula | Formula | Formula | weight | By |
| Classification | Weight | Purity | oxide | 2a | 2b | 2c | (g)^{a)} | purity⁾ |
| Crude | (100 g) | 91.28% | 2.56% | 3.33% | 0.62% | 1.46% | — | — |
| simvastatin | | | | | | | | |
| Crystal #1 | 93.3 g | 96.86% | 1.55% | 0.39% | 0.52% | 0.52% | 93.3% | 99.0% |
| Crystal #2 | 83.7 g | 99.15% | 0.20% | 0.04% | 0.39% | 0.16% | 89.0% | 91.2% |
| Simvastatin | 82.0 g | 99.32% | 0.05% | 0.04% | 0.38% | 0.15% | 98.0% | 98.2% |

^{a)}Yield of simvastatin obtained from 100 g of crude simvastatin.
^{b)}Yield calculated by considering purities of crude simvastatin and simvastatin crystals.

As seen in Table 1, the total yield of the simvastatin crystal obtained by the present invention was 82.0%, and the total yield calculated by considering the purity of crude simvastatin was 89.2%.

### Example 2

100 g of crude simvastatin was dissolved in 650 ml of tetrahydrofuran and completely concentrated at about 15° C. During the concentration, simvastatin was added in as a seed for crystallization. 3000 ml of diethyl ether was added for 30 minutes while vigorously stirring the solution at 15° C. Then, the solution was slowly cooled to 5° C. for 1 hour. This solution was stirred for 1 hour at 5° C., and then filtered and dried to obtain 91g of Crystal #1.

The obtained 91 g of Crystal #1 was suspended in 350 ml of isopropyl acetate. Then, 1400 ml of diethyl ether was dropped for 30 minutes and the temperature was raised to 30° C. The solution was cooled to 5° C. for 1 hour, stirred at the same temperature for 1 hour, and then filtered. The filtration mother liquor was collected and dried. The crystal was washed with hexane and dried to obtain 77.35 g of Crystal #2.

The obtained 77.35 g of Crystal #2 was dissolved in 350 ml of 1,4-dioxane. After adding 4.3 g of active carbon, the solution was stirred at 20° C. for 30 minutes and filtered. The filtrate was washed with 88 ml of 1,4-dioxane and 1300 ml of water was dropped for 1 hour at 20° C. After stirring for 1 hour at the same temperature, it was washed with 1700 ml of water. Then, it was washed with hexane and dried to obtain 75.0 g of high-purity simvastatin.

Each crystal obtained in each crystallization step was analyzed with an HPLC (Waters; 2690). The content analysis result is shown in the following Table 2.

TABLE 2

| | | | | Impurities | | | | Yield | |
|---|---|---|---|---|---|---|---|---|---|
| | | | | | Chemical | Chemical | Chemical | By | |
| | Simvastatin | | | Triphenylphosphine | Formula | Formula | Formula | weight | By |
| Classification | Weight | | Purity | oxide | 2a | 2b | 2c | (g)^{a)} | purity⁾ |
| Crude | (100 | g) | 91.28% | 2.56% | 3.33% | 0.62% | 1.46% | — | — |
| simvastatin | | | | | | | | | |
| Crystal #1 | 91 | g | 96.21% | 1.48% | 0.45% | 0.59% | 0.53% | 91.0% | 95.9% |
| Crystal #2 | 77.35 | g | 99.01% | 0.19% | 0.05% | 0.35% | 0.15% | 85.0% | 87.4% |
| Simvastatin | 75.0 | g | 99.12% | 0.04% | 0.04% | 0.34% | 0.15% | 97.0% | 97.1% |

^{a)}Yield of simvastatin obtained from 100 g of crude simvastatin.
^{b)}Yield calculated by considering purities of crude simvastatin and simvastatin crystals.

As described above, the present invention effectively removes impurities contained in crude simvastatin obtained in the course of simvastatin synthesis while minimizing the loss of acquired simvastatin, thus being useful for obtaining high-purity simvastatin.

While the present invention has been described in detail with reference to the preferred embodiments, those skilled in the art will appreciate that various modifications and substitutions can be made thereto without departing from the spirit and scope of the present invention as set forth in the appended claims.

What is claimed is:

1. A purification method of crude simvastatin comprising:
(a) a step of concentrating crude simvastatin by dissolving in a solvent, adding simvastatin seed in said concentrate for partial crystallization and adding hexane or cyclohexane to obtain Crystal #1;
(b) a step of suspending said Crystal #1 in alkyl acetate and adding hexane or cyclohexane to obtain Crystal #2; and
(c) a step of dissolving said Crystal #2 in a solvent selected from a group consisting of dimethoxyethane, tetrahydrofuran, 1,4dioxane, dimethylether and dimethoxypropane and adding purified water to obtain purified simvastatin crystal.

2. The purification method according to claim 1, wherein said crude simvastatin contains as impurities a compound represented by the following Chemical Formula 2a, 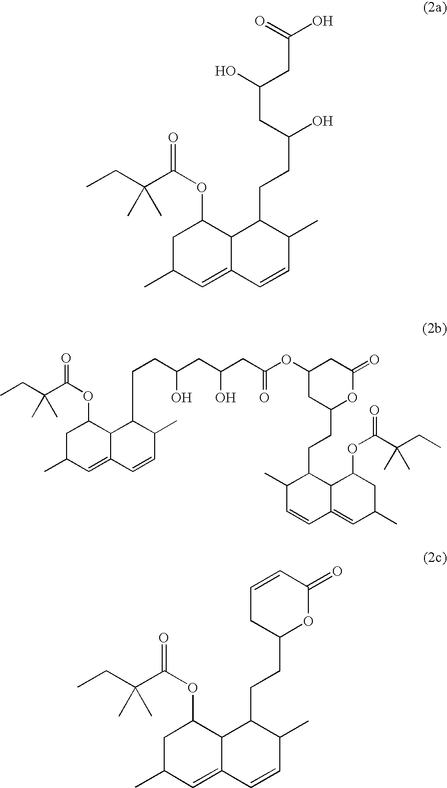
which is generated by opening of the lactone ring, or a dimer represented by the following Chemical Formula 2b, or anhydrosimvastatin represented by the following Chemical Formula 2c or a mixture of these thereof.

3. The purification method according to claim 2, wherein said crude simvastatin contains as impurities more than 3% of the compound represented by Chemical Formula 2a, which is generated by opening of the lactone ring, or more than 0.6% of the dimer represented by Chemical Formula 2b, or more than 1% of anhydrosimvastatin represented by Chemical Formula 2c or a mixture of these thereof.

4. The purification method according to any one of claims 1 to 3, wherein said crude simvastatin further contains more than 2% of triphenylphosphine oxide as an impurity.

5. The purification method according to claim 1, wherein said solvent dissolving said crude simvastatin is selected from a group consisting of methylene chloride, chloroform, tetrahydrofuran and ethyl acetate.

6. The purification method according to claim 1, wherein the temperature of dissolving said crude simvastatin in step (a) is maintained in the range of 0 to 40° C.

7. The purification method according to claim 1, wherein said temperature of suspending said crude simvastatin in step (b) is maintained in the range of 0 to 40° C.

8. The purification method according to claim 1, wherein said purified water at 10 to 30° C. is added for 30 minutes −3 hours in step (c).

9. The purification method according to claim 1, wherein a filtration process using active carbon is carried out prior to adding purified water to said Crystal #2 in step (c).

10. The purification method according to claim 1, wherein solutions remaining from steps (a), (b) and (c) are collected, concentrated and added to said crude simvastatin in said step (a).

11. Simvastatin containing less than 0.1% of a compound represented by Chemical Formula 2a, which is generated by opening of the lactone ring, less than 0.4% of a dimer represented by the following Chemical Formula 2b, and less than 0.4% of anhydrosimvastatin represented by the following Chemical Formula 2c as impurities.
